(19) United States
(12) Patent Application Publication
(10) Pub. No.: US 2009/0089919 A1
(43) Pub. Date: Apr. 9, 2009

(54) Female urination device

(76) Inventor: Cynthia K. Rudolph, Strongsville, OH (US)

Correspondence Address: James A. Hudak, Esq.;Suite #304, 29425 Chagrin Blvd., Cleveland, OH, 44122, US

(21) Appl. No.: 11/973,213

(22) Filed: Oct. 9, 2007

(51) Int. Cl. A47K 11/12; A61M 1/00

(52) U.S. Cl. 4/144.4; 4/144.1; 604/317

(57) ABSTRACT

A urination device that permits a woman to direct her urine into a toilet while in the standing position is disclosed. The device is generally funnel-shaped in configuration with an elongated lip portion at its entrance and is formed from biodegradable material so as to be readily disposable. The exposed edges of the device are rolled to prevent the creation of any discomfort or irritation when the device is used. An absorbent material is attached to the outer surface of the device for personal wiping purposes. The elongated lip portion can be folded onto the funnel-shaped portion creating a compact unit for carrying purposes.

### TECHNICAL FIELD

The present invention relates, in general, to a urination device and, more particularly, to a urination device which permits a woman to urinate into a toilet while in the standing position.

### BACKGROUND ART

Public restrooms for women are often unclean and unsanitary. In particular, the toilet seats in public restrooms are often dirty or otherwise in an unsanitary condition. Because of this situation, many women are required to place themselves in a sitting position above the toilet seat when using such restrooms so as to avoid contact with a dirty or unsanitary toilet seat. This is a very uncomfortable and unpleasant position for a woman to be in when urinating. Similarly, when camping or traveling, a ladies restroom may not be readily available necessitating that a woman assume a squatting position when urinating while holding her clothing out of the way.

In view of the foregoing inherent problems typically associated with ladies restrooms, it is desirable to develop a urination device which permits a woman to urinate into a toilet while in the standing position and which is simple in construction, easy to use and readily disposable.

### SUMMARY OF THE INVENTION

The present invention is directed to a urination device that permits a woman to direct her urine into a toilet while in the standing position. The device is generally funnel-shaped in configuration with an elongated lip portion at its inlet and has a waterproof inner surface. The outer surface of the device has an absorbent material, similar to toilet paper, attached thereto for wiping purposes. The device is formed from biodegradable material so as to be readily disposable. The exposed edges of the device are rolled so as to be similar to the top surface of a paper-drinking cup. The outer edges where the funnel-shaped portion joins the elongated lip portion are rounded. The elongated lip portion can be folded onto the funnel-shaped portion creating a compact unit. The device can be fabricated from a single piece of material resulting in only one seam for joining purposes.

### BRIEF DESCRIPTION OF THE DRAWINGS

FIG. 1 is a top plan view of the paper blank that is utilized to form the female urination device of the present invention.

FIG. 2 is a perspective view of the female urination device of the present invention.

FIG. 3 is a front elevational view of the female urination device of the present invention.

FIG. 4 is a side elevational view of the female urination device of the present invention.

FIG. 5 is a cross-sectional view taken across section indicating lines 5-5 in FIG. 4 and illustrates the rounded edge at the inlet to the female urination device of the present invention.

FIG. 6 is a cross-sectional view taken across section indicating lines 6-6 in FIG. 4 and illustrates the single seam used to join the funnel-shaped portion of the female urination device of the present invention.

### DESCRIPTION OF THE PREFERRED EMBODIMENT

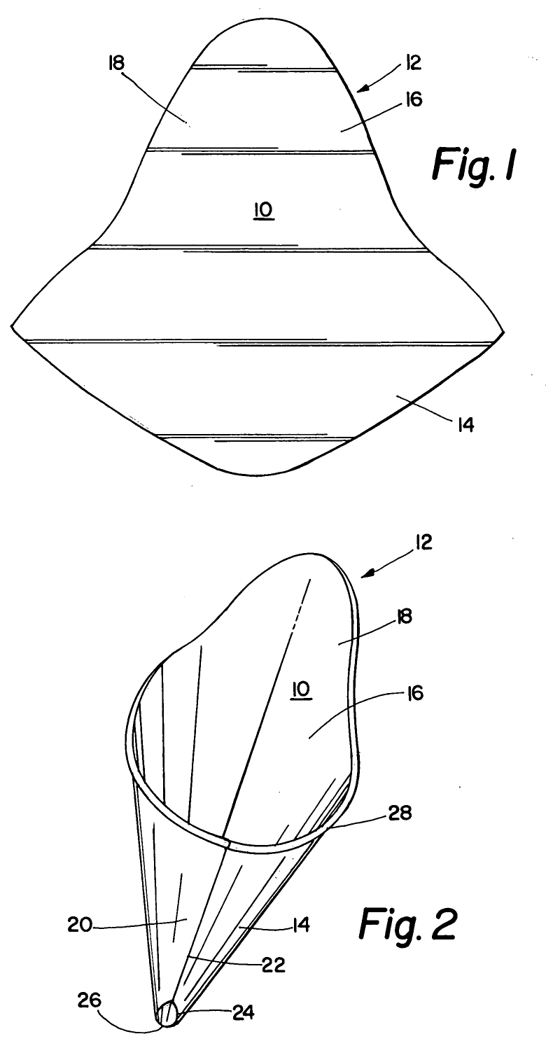

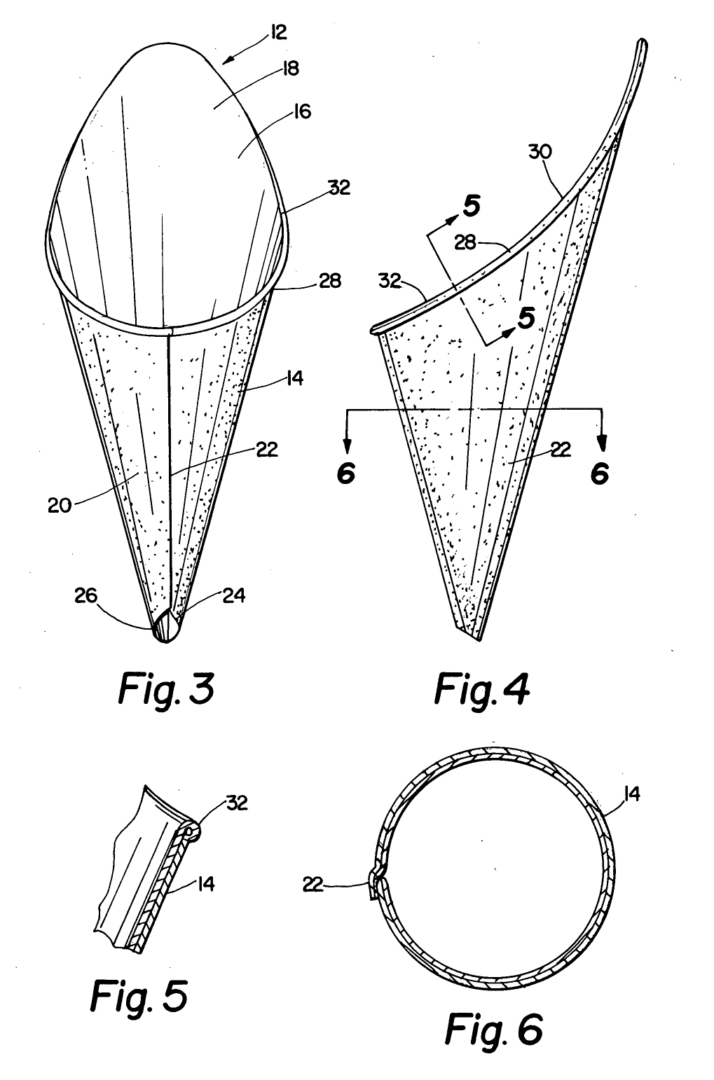

Referring now to the drawings where the illustrations are for the purpose of describing the preferred embodiment of the present invention and are not intended to limit the invention disclosed herein, FIG. 1 is a top plan view of the blank 10 that is utilized to form the female urination device 12 of the present invention. The blank 10 is made from biodegradable material and has a configuration generally as illustrated. As such, the blank 10 has a first portion 14 which is formed into a generally funnel-shaped configuration and a second portion 16 which provides an elongated lip 18 at the entrance to the funnel-shaped portion. The reverse side of the blank 10 has an absorbent material 20 (not shown in FIG. 1) for personal wiping purposes. The absorbent material 20 is similar to toilet paper, or the like.

Forming the first portion 14 of the blank 10 into a generally funnel-shaped configuration results in the female urination device 12 of the present invention having an overall shape as illustrated in FIG. 2. The forming of the blank 10 into a funnel-shaped configuration results in the creation of a single seam 22, shown in cross-section in FIG. 6, where the oppositely disposed edges of the first portion 14 of the blank 10 are joined and an aperture 24 in the outlet 26 of the first portion 14. Forming of the blank 10 also causes the second portion 16, which defines the elongated lip 18 to extend outwardly from the entrance 28 to the funnel-shaped first portion 14 and to have a substantially semi-circular configuration in cross-section. The elongated lip 18 provides a guiding surface for the urine stream, which passes through the funnel-shaped portion 14 to the aperture 24 in the outlet 26 thereof After forming, the absorbent material 20 is on the exterior surface of the urination device 12.

As shown in FIG. 4 which is a side elevational view of the female urination device 12 of the present invention, the inlet 30 to the device 12 is curved so as to fit the female anatomy and has a rolled edge 32, shown in cross-section in FIG. 5, so as to prevent the creation of any discomfort or irritation when the urination device 12 is used. After use, the absorbent material 20 on the exterior of the urination device 12 is used for personal wiping purposes. It should be noted that the elongated lip portion 18 can be folded onto the funnel-shaped portion 14 to create a compact unit for carrying purposes.

Certain modifications and improvements will occur to those skilled in the art upon reading the forgoing. It is understood that all such modifications and improvements have been deleted herein for the sake of conciseness and readability, but are properly within the scope of the following claims.

1) A female urination device comprising a first portion having an inlet and an outlet and a second portion operatively attached to said inlet to said first portion, said first portion having a substantially funnel-shape configuration in cross-section and said second portion having a substantially semi-circular configuration in cross-section.

2) The device as defined in claim 1 wherein said first and second portion are formed from a unitary blank of material.

3) The device as defined in claim 2 wherein said material is biodegradable.

4) The device as defined in claim 1 wherein said second portion is elongated.

5) The device as defined in claim 1 wherein said second portion has a rolled edge substantially around the periphery thereof

6) The device as defined in claim 1 further including an absorbent material attached to the outer surface of said device.
